(19) United States
(12) Patent Application Publication
(10) Pub. No.: US 2025/0221645 A1
(43) Pub. Date: Jul. 10, 2025

(54) APPARATUS AND METHOD FOR IDENTIFYING COLLATERAL PROCESSES

(71) Applicant: The Strategic Coach Inc., Toronto (CA)

(72) Inventors: Barbara Sue Smith, Toronto (CA); Daniel J. Sullivan, Toronto (CA)

(73) Assignee: The Strategic Coach Inc., Toronto, ON (CA)

(21) Appl. No.: 18/406,573

(22) Filed: Jan. 8, 2024

(51) Int. Cl. A61B 5/16

(52) U.S. Cl. CPC A61B 5/165; A61B 5/163; G06N 3/0985; A61B 3/10; A61B 3/113; A61B 5/11; A61B 5/16; A61B 5/4088; A61N 1/36082; G01N 2800/2814; G02B 27/0093; G05B 2219/34075; G06F 3/013; G06N 3/02; G06N 3/06; G06N 3/08; G06N 20/00; G06N 20/10; G06N 20/20; G06N 99/00; G16H 50/70; H04L 41/16

(57) ABSTRACT

An apparatus and method identifies collateral processes. [T]he apparatus including a processor configured to measure a plurality of process data of a process, generate a first process model using the plurality of process data, receive a plurality of metamodel training examples, train, using the plurality of metamodel training examples, a metamodel, generate a measurement of the first process model and output a model efficiency score of the first process model using the metamodel and the measurement of the first process model.

### FIELD OF THE INVENTION

The present invention generally relates to the field of automated cognitive assessment. In particular, the present invention is directed to identifying collateral processes.

### BACKGROUND

Machine-learning models tend to be locally optimized to solutions presented by training examples and some random variation in stochastic algorithms or initial conditions. It is possible, however, for a model to be globally suboptimal. This can be difficult to detect using automated systems.

### SUMMARY OF THE DISCLOSURE

In an aspect an apparatus for identifying collateral processes, the apparatus including at least a processor and a memory communicatively connected to the at least a processor, the memory containing instructions configuring the processor to measure a plurality of process data of a process, where the plurality of process data further includes a plurality of process input data and a plurality of correlated process output data and generate a first process model using the plurality of process data, wherein generating the first process model further includes training the process model using the plurality of process input data and the plurality of correlated process output data. The processor further configured to receive a plurality of metamodel training examples, wherein each metamodel training example includes a plurality of model input examples and model output examples corresponding to an exemplary model, and wherein each metamodel training example further includes at least an efficiency score relating to the plurality of model input examples and model output examples, train, using the plurality of metamodel training examples, a metamodel, wherein the metamodel is configured to receive process model measurements and output model efficiency scores, generate a measurement of the first process model and output a model efficiency score of the first process model using the metamodel and the measurement of the first process model.

In another aspect a method for identifying collateral processes, where the method includes measuring a plurality of process data of a process, wherein the plurality of process data further includes a plurality of process input data and a plurality of correlated process output data, generating a first process model using the plurality of process data, wherein generating the first process model further includes training the process model using the plurality of process input data and the plurality of correlated process output data. The method also including receiving a plurality of metamodel training examples, wherein each metamodel training examples includes a plurality of model input examples and model output examples corresponding to an exemplary model, and wherein each metamodel training example further includes at least an efficiency score relating to the plurality of model input examples and model output examples, training a metamodel using the plurality of metamodel training examples, wherein the metamodel is configured to receive process model measurements and output model efficiency scores, generating a measurement of the first process model and outputting a model efficiency score of the first process model using the metamodel and the measurement of the first process model.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for the identifying collateral processes;

FIG. 2 is a diagram of an exemplary embodiment of a user interface;

FIG. 3 is a block diagram of an exemplary machine-learning process;

FIG. 4 is a diagram of an exemplary embodiment of a neural network;

FIG. 5 is a diagram of an exemplary embodiment of a node of a neural network;

FIG. 6 is an illustration of an exemplary embodiment of fuzzy set comparison;

FIG. 7 is an illustration of an exemplary embodiment of a chatbot;

FIG. 8 is a flow diagram of an exemplary method for the identifying collateral processes; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for identifying collateral processes.

Aspects of the present disclosure allow for identifying causative factors based on complexity and severity of an issue. Exemplary embodiments illustrating aspects of the present disclosure are described below in the context of several specific examples.

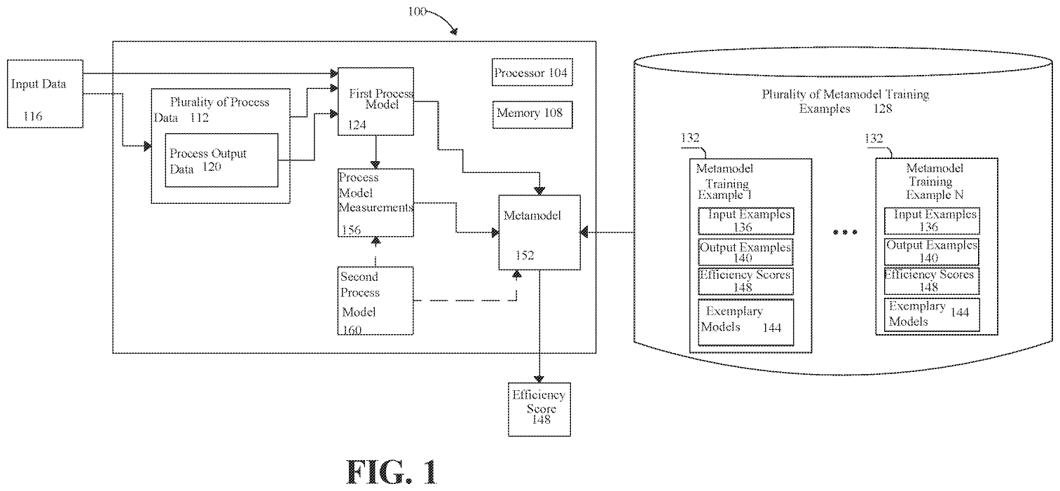

Referring now to FIG. 1, an exemplary embodiment of an apparatus 100 for identifying causative factors of cognitive distress is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Processor 104 may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or processor 104.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Continuing to refer to FIG. 1, in an embodiment, methods and apparatus described herein may perform or implement one or more aspects of a cryptographic system. In one embodiment, a cryptographic system is a system that converts data from a first form, known as “plaintext,” which is intelligible when viewed in its intended format, into a second form, known as “ciphertext,” which is not intelligible when viewed in the same way. Ciphertext may be unintelligible in any format unless first converted back to plaintext. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that arc “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

Still referring to FIG. 1, in embodiments of cryptographic systems that are “asymmetric,” cither encryption or decryption key cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. An example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. Another example is elliptic curve cryptography, which relies on the fact that given two points P and Q on an elliptic curve over a finite field, and a definition for addition where A+B=−R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q.

Still referring to FIG. 1, in some embodiments, apparatus and methods described herein produce cryptographic hashes, also referred to by the equivalent shorthand term “hashes.” A cryptographic hash, as used herein, is a mathematical representation of a lot of data, such as files or blocks in a block chain as described in further detail below; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is a one-way function, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

With continued reference to FIG. 1, in an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running the lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, SHA256, a NIST standard; further current and past hashing algorithms include Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-512,” and the like), Message Authentication Code (“MAC”)-family hash functions such as PMAC, OMAC, VMAC, HMAC, and UMAC, Poly 1305-AES, Elliptic Curve Only Hash (“ECOH”) and similar hash functions, Fast-Syndrome-based (FSB) hash functions, GOST hash functions, the Grøstl hash function, the HAS-160 hash function, the JH hash function, the RadioGatun hash function, the Skein hash function, the Strecbog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” may to detect collision may be on the order of O(2^{n/2}) for n output bits; thus, it may take on the order of 2²⁵⁶ operations to locate a collision in a 512 bit output “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

Continuing to refer to FIG. 1, a “secure proof,” as used in this disclosure, is a protocol whereby an output is generated that demonstrates possession of a secret, such as device-specific secret, without demonstrating the entirety of the device-specific secret; in other words, a secure proof by itself, is insufficient to reconstruct the entire device-specific secret, enabling the production of at least another secure proof using at least a device-specific secret. A secure proof may be referred to as a “proof of possession” or “proof of knowledge” of a secret. Where at least a device-specific secret is a plurality of secrets, such as a plurality of challenge-response pairs, a secure proof may include an output that reveals the entirety of one of the plurality of secrets, but not all of the plurality of secrets; for instance, secure proof may be a response contained in one challenge-response pair. In an embodiment, proof may not be secure; in other words, proof may include a one-time revelation of at least a device-specific secret, for instance as used in a single challenge-response exchange.

Still referring to FIG. 1, secure proof may include a zero-knowledge proof, which may provide an output demonstrating possession of a secret while revealing none of the secret to a recipient of the output; zero-knowledge proof may be information-theoretically secure, meaning that an entity with infinite computing power would be unable to determine secret from output. Alternatively, zero-knowledge proof may be computationally secure, meaning that determination of secret from output is computationally infeasible, for instance to the same extent that determination of a private key from a public key in a public key cryptographic system is computationally infeasible. Zero-knowledge proof algorithms may generally include a set of two algorithms, a prover algorithm, or “P,” which is used to prove computational integrity and/or possession of a secret, and a verifier algorithm, or “V” whereby a party may check the validity of P. Zero-knowledge proof may include an interactive zero-knowledge proof, wherein a party verifying the proof must directly interact with the proving party; for instance, the verifying and proving parties may be required to be online, or connected to the same network as each other, at the same time. Interactive zero-knowledge proof may include a “proof of knowledge” proof, such as a Schnorr algorithm for proof on knowledge of a discrete logarithm. in a Schnorr algorithm, a prover commits to a randomness r, generates a message based on r, and generates a message adding r to a challenge c multiplied by a discrete logarithm that the prover is able to calculate; verification is performed by the verifier who produced c by exponentiation, thus checking the validity of the discrete logarithm. Interactive zero-knowledge proofs may alternatively or additionally include sigma protocols. Persons skilled in the art, upon reviewing the entirety of this disclosure, will be aware of various alternative interactive zero-knowledge proofs that may be implemented consistently with this disclosure.

Alternatively, and still referring to FIG. 1, zero-knowledge proof may include a non-interactive zero-knowledge, proof, or a proof wherein neither party to the proof interacts with the other party to the proof; for instance, each of a party receiving the proof and a party providing the proof may receive a reference datum which the party providing the proof may modify or otherwise use to perform the proof. As a non-limiting example, zero-knowledge proof may include a succinct non-interactive arguments of knowledge (ZK-SNARKS) proof, wherein a “trusted setup” process creates proof and verification keys using secret (and subsequently discarded) information encoded using a public key cryptographic system, a prover runs a proving algorithm using the proving key and secret information available to the prover, and a verifier checks the proof using the verification key; public key cryptographic system may include RSA, elliptic curve cryptography, ElGamal, or any other suitable public key cryptographic system. Generation of trusted setup may be performed using a secure multiparty computation so that no one party has control of the totality of the secret information used in the trusted setup; as a result, if any one party generating the trusted setup is trustworthy, the secret information may be unrecoverable by malicious parties. As another non-limiting example, non-interactive zero-knowledge proof may include a Succinct Transparent Arguments of Knowledge (ZK-STARKS) zero-knowledge proof. In an embodiment, a ZK-STARKS proof includes a Merkle root of a Merkle tree representing evaluation of a secret computation at some number of points, which may be 1 billion points, plus Merkle branches representing evaluations at a set of randomly selected points of the number of points; verification may include determining that Merkle branches provided match the Merkle root, and that point verifications at those branches represent valid values, where validity is shown by demonstrating that all values belong to the same polynomial created by transforming the secret computation. In an embodiment, ZK-STARKS does not require a trusted setup.

Continuing to refer to FIG. 1, zero-knowledge proof may include any other suitable zero-knowledge proof. Zero-knowledge proof may include, without limitation, bulletproofs. Zero-knowledge proof may include a homomorphic public-key cryptography (hPKC)-based proof. Zero-knowledge proof may include a discrete logarithmic problem (DLP) proof. Zero-knowledge proof may include a secure multi-party computation (MPC) proof. Zero-knowledge proof may include, without limitation, an incrementally verifiable computation (IVC). Zero-knowledge proof may include an interactive oracle proof (IOP). Zero-knowledge proof may include a proof based on the probabilistically checkable proof (PCP) theorem, including a linear PCP (LPCP) proof. Persons skilled in the art, upon reviewing the entirety of this disclosure, will be aware of various forms of zero-knowledge proofs that may be used, singly or in combination, consistently with this disclosure.

Still referring to FIG. 1, in an embodiment, secure proof is implemented using a challenge-response protocol. In an embodiment, this may function as a one-time pad implementation; for instance, a manufacturer or other trusted party may record a series of outputs (“responses”) produced by a device possessing secret information, given a series of corresponding inputs (“challenges”), and store them securely. In an embodiment, a challenge-response protocol may be combined with key generation. A single key may be used in one or more digital signatures as described in further detail below, such as signatures used to receive and/or transfer possession of crypto-currency assets; the key may be discarded for future use after a set period of time. In an embodiment, varied inputs include variations in local physical parameters, such as fluctuations in local electromagnetic fields, radiation, temperature, and the like, such that an almost limitless variety of private keys may be so generated. Secure proof may include encryption of a challenge to produce the response, indicating possession of a secret key. Encryption may be performed using a private key of a public key cryptographic system, or using a private key of a symmetric cryptographic system; for instance, trusted party may verify response by decrypting an encryption of challenge or of another datum using either a symmetric or public-key cryptographic system, verifying that a stored key matches the key used for encryption as a function of at least a device-specific secret. Keys may be generated by random variation in selection of prime numbers, for instance for the purposes of a cryptographic system such as RSA that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem. Keys may be generated by random variation in selection of prime numbers, for instance for the purposes of a cryptographic system such as secret that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem.

With continued reference to FIG. 1, a “digital signature,” as used herein, includes a secure proof of possession of a secret by a signing device, as performed on provided element of data, known as a “message.” A message may include an encrypted mathematical representation of a file or other set of data using the private key of a public key cryptographic system. Secure proof may include any form of secure proof as described above, including without limitation encryption using a private key of a public key cryptographic system as described above. Signature may be verified using a verification datum suitable for verification of a secure proof; for instance, where secure proof is enacted by encrypting message using a private key of a public key cryptographic system, verification may include decrypting the encrypted message using the corresponding public key and comparing the decrypted representation to a purported match that was not encrypted; if the signature protocol is well-designed and implemented correctly, this means the ability to create the digital signature is equivalent to possession of the private decryption key and/or device-specific secret. Likewise, if a message making up a mathematical representation of file is well-designed and implemented correctly, any alteration of the file may result in a mismatch with the digital signature; the mathematical representation may be produced using an alteration-sensitive, reliably reproducible algorithm, such as a hashing algorithm as described above. A mathematical representation to which the signature may be compared may be included with signature, for verification purposes; in other embodiments, the algorithm used to produce the mathematical representation may be publicly available, permitting the easy reproduction of the mathematical representation corresponding to any file.

Still viewing FIG. 1, in some embodiments, digital signatures may be combined with or incorporated in digital certificates. In one embodiment, a digital certificate is a file that conveys information and links the conveyed information to a “certificate authority” that is the issuer of a public key in a public key cryptographic system. Certificate authority in some embodiments contains data conveying the certificate authority's authorization for the recipient to perform a task. The authorization may be the authorization to access a given datum. The authorization may be the authorization to access a given process. In some embodiments, the certificate may identify the certificate authority. The digital certificate may include a digital signature.

With continued reference to FIG. 1, in some embodiments, a third party such as a certificate authority (CA) is available to verify that the possessor of the private key is a particular entity; thus, if the certificate authority may be trusted, and the private key has not been stolen, the ability of an entity to produce a digital signature confirms the identity of the entity and links the file to the entity in a verifiable way. Digital signature may be incorporated in a digital certificate, which is a document authenticating the entity possessing the private key by authority of the issuing certificate authority and signed with a digital signature created with that private key and a mathematical representation of the remainder of the certificate. In other embodiments, digital signature is verified by comparing the digital signature to one known to have been created by the entity that purportedly signed the digital signature; for instance, if the public key that decrypts the known signature also decrypts the digital signature, the digital signature may be considered verified. Digital signature may also be used to verify that the file has not been altered since the formation of the digital signature.

Continuing to refer to FIG. 1, apparatus 100 includes a memory 108 communicatively connected to the at least a processor 104, wherein the memory 108 contains instructions configuring processor 104 to perform tasks in accordance with this disclosure. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, in an embodiment, processor 104 is configured to measure a plurality of process data 112 of a process, wherein the plurality of process data 112 further comprises a plurality of process input data 116 and a plurality of correlated process output data 120. As used herein, “process data” is data collected and recorded. In embodiments, process data 112 may include input data, control data, operational data, output data, time stamps, sensor data, log file data, metadata, and the like. “Process input data,” as used herein, is measurable data received from an external source. In some embodiments, process input data may include signals from one or more sensors. In an embodiment, process input data may include sensor data. In some embodiments, process input data may include a user input. In embodiments, process input data may include textual inputs, numerical data, image data, video data, audio data, timeseries data, geospatial data, binary data, structured data, and the like. A “process output data,” as used herein, is data generated as an output from a system, apparatus or process after performing operations, calculation or transformations on input data.

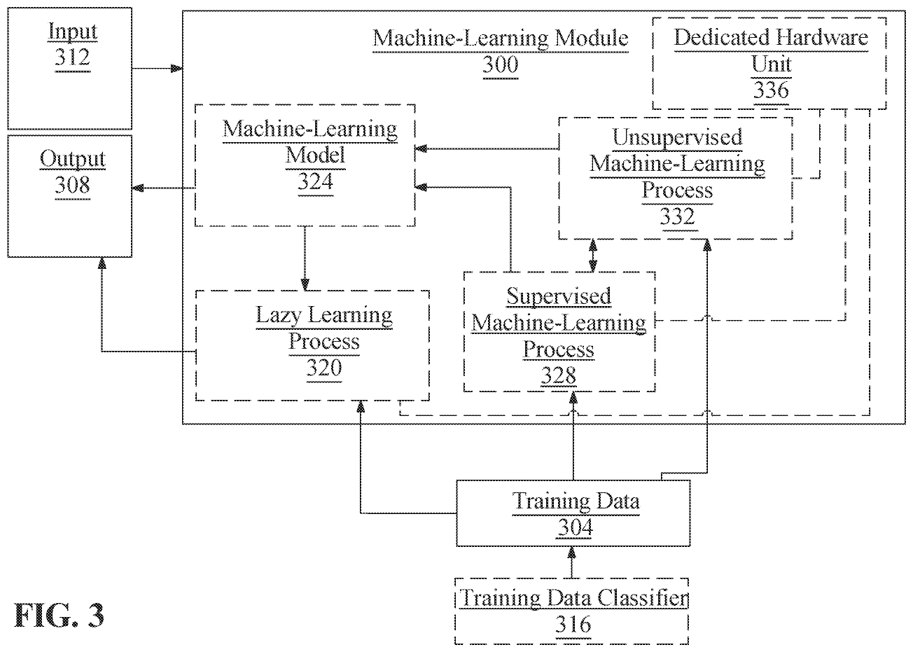

Continuing to refer to FIG. 1, in some embodiments, processor 104 is configured to generate a first process model 124 using the plurality of process data 112. A “first process model, as used herein, is a data structure that represents or emulates behavior of a corresponding process. In embodiments, generating the first process model 124 may further include training first process model 124 using the plurality of process input data 116 and plurality of correlated process output data 120. In an embodiment, first process model 124 may be a machine learning model. In embodiments, first process model 124 may be a neural network. In some embodiments, first process model 124 may receive input data 116 as input. In some embodiments, processor 104 may be configured to generate an error metric for first process model 124 by comparing output of first process model 124 to process output data 120. An “error metric,” as used herein, is a metric describing the discrepancy between the output generated by a model and an expected known output. First process model 124 may include any model and/or algorithm described in reference to FIGS. 3-6.

Still referring to FIG. 1, in an embodiment, processor 104 is configured to receive a plurality of metamodel training examples 128. In embodiments, each metamodel training example 132 may include a plurality of model input examples 136 and model output examples 140 corresponding to an exemplary model 144. In some embodiments, each metamodel training example 132 may further include at least a model efficiency score 148 relating to the plurality of model input examples 136 and model output examples 140. In nonlimiting examples, exemplary model efficiency scores 148 may include efficiency scores generated for previous models. In embodiments, exemplary models 144 may include previously generated models. In some embodiments, exemplary models 144, model input examples 136, model output examples 140 may include mock data. As used herein, mock data,” is exemplary data input and/or generated for the purpose of training one or more models. In some embodiments, model efficiency score 148 for an exemplary model 144 may be generated based on error metrics generated by comparing model output examples 140 to expected outputs. In some embodiments, plurality of metamodel training examples 128 may include historical data. In embodiments, historical data may include error metrics generated for each metamodel training example 132. In some embodiments, historical data may include error metrics generated a previous iterations of models used as exemplary models 144. In some embodiments, historical data may include mock error metrics for exemplary models 144, such as data input by a user for the purpose of training metamodels. Efficiency score 148 is described in more detail below. Exemplary models 144 may include any model described in reference to FIGS. 3-6.

With continued reference to FIG. 1, in an embodiment, processor 104 is configured to train, using plurality of metamodel training examples 128, a metamodel 152, wherein metamodel 152 is configured to receive process model measurements 156 as input and output model efficiency scores 148. An “efficiency score,” as used herein, is the efficiency or effectiveness of a model based on their performance in relation to one or more metrics. In embodiments, metrics may include mean squared error (MSE), Mean Absolute Error (MAE), R-squared, Accuracy of classifications, Precision and Recall, F1-score, Computational Efficiency, Median Absolute Deviation (MAD), and the like. Metamodel 152 is described in more detail below. In some embodiments, process model measurements 156 may include one or more metrics generated based on a comparison between process output data 120 and output of first process model 124. In embodiments efficiency scores based on metrics may be performed using a plurality of approaches such as linear mapping, threshold-based approach, precent improvement, ranking, weighted combination, muti-criteria decision making, and the like. In some embodiments, metric to be used may be defined by a user. In embodiments, metamodel 152 may be configured to normalize the metrics. In embodiments, metamodel 152 may utilize a plurality of normalization techniques, such as Min-Max Scaling, Standardization, Decimal Scaling, Log Transformation, Robust Scaling, and the like. In some embodiments, metamodel 152 may be configured to use the same metrics used for calculating efficiency scores 148 in plurality of metamodel training examples 128. In a nonlimiting example, metamodel 152 may use Mean Absolute Error (MAE) as a metric. In this nonlimiting example, the MAE target may be preset to a threshold, in this case a threshold of 5 or below for the MAE. Continuing on this nonlimiting example, because MAE metrics are absolute value, no normalization of the metrics is necessary. Still on this nonlimiting example, metamodel 152 may be configured to use linear mapping for calculating the efficiency score based on the MAE value. Linear mapping of MAE may be described as Efficiency Score=max (0,1−(MAE/Target MAE)). In this nonlimiting example, processor 104 may have generated a MAE of 6 based on the comparison between process output data 120 and output of first process model 124. Continuing on this nonlimiting example, metamodel 152 may apply the formula above to derive the efficiency score, which may be described as Efficiency Score=max(0,1−(6/5)=max(0,1−1.2)=max(0, −0.2)=0. In this nonlimiting example, the efficiency score of 0 indicates that first process model 124 is operating with an error rate above a set threshold, thus indicating that further optimization of the model is necessary.

Continuing to refer to FIG. 1, in an embodiment, processor 104 is configured to generate a measurement 156 of the first process model 124. A “measurement,” as used herein, is one or more metrics generated based on the comparison between a model output and an expected output. In embodiments, measurement 156 may include performance measurement, accuracy measurement, security measurement, reliability measurement, robustness measurement, compliance measurement, usability measurement, and the like. In some embodiments, processor 104 may be configured to apply cryptographic techniques to measurement 156, such as cryptographic hashing and digital signatures. In a nonlimiting example, processor 104 may be configured to generate a hash value for measurement 156, using a hash function, and verify integrity of the measurement at a later time by recalculating the hash value using the hash function. Processor 104 may be configured to apply, or use, any cryptographic technique and systems described throughout this disclosure. Cryptographic methods and systems are described in more detail below.

Still referencing to FIG. 1, in embodiments, processor 104 is configured to output a model efficiency score 148 of the first process model 124 using metamodel 152 and the measurement 156 of the first process model 124. In some embodiments, processor 104 may be configured to generate a second process model 160. In a nonlimiting example, metamodel 152 may be configured to generate efficiency score related to robustness measurement, such a Median Absolute Deviation (MAD) metric generated based on a comparison of first process model 124 output and process output data 120. In this nonlimiting example, metamodel 152 may apply a normalization technique, described further above, to MAD metric, so as to ensure MAD metric is in a common scale with other comparable metrics. Continuing on this nonlimiting example, metamodel 152 may be configured to define the efficiency score calculation as the inverse of the normalized robustness metric, where this calculation maps a higher robustness to a lower efficiency score, which indicates that a lower dispersion is desirable for a higher efficiency score. Calculation formula may be described as Efficiency Score=1−Normalized MAD. In this nonlimiting example, processor 104 may generate a metric based on a comparison of first process model 124 and process output data 120, where it may be equal to 0.3 after normalization is applied. In this nonlimiting example, metamodel 152 may derive an efficiency score of 0.7, which may indicate a good level of robustness depending on use case. Calculation may be described as Efficiency Score=1−0.3=0.7.

Continuing to refer to FIG. 1, in embodiments, processor 104 may be configured to compare first process model 124 to second process model 160 using metamodel 152. In an embodiment, processor 104 may generate an efficiency score 148 for first process model 124 and an efficiency score 148 for second process model 160 using metamodel 152. In some embodiments, processor 104 may be configured to compare efficiency score 148 of first process model 124 to efficiency score 148 of second process model 160. In embodiments, processor 104 may be configured to compare the output of first process model 124 to the output of second process model 160. In some embodiments, processor 104 may be configured to compare efficiency score 148 of first process model 124 to efficiency score 148 for second process model 160. In further embodiments, processor 104 may select between first process model 124 and second process model 160 based on the comparison. In embodiments, processor 104 may be further configured to generate a measurement of second process model 160. In an embodiment, metamodel 152 may receive process model measurement 156 of first process model 124 and process model measurement 156 of second process model 160 as inputs and output an efficiency score. In a nonlimiting example, processor 104 may use a process model measurement 156 of first process model 124, such as accuracy metric, and a process model measurement 156 of second process model 160, such as precision. In this nonlimiting example, processor 104 may normalize the metrics into a common scale before combining the metrics. Continuing with this nonlimiting example, processor 104 may be configured to assign weighting factors to each metric relative to their importance. Weighting factor may be based on preset rules. Weighting factor may be based on expected use of models, where in instances where minimizing an error metric is more important, a higher weight may be given to an accuracy metric, while a higher weight may be given to precision when generating outputs within a range of correctness is more important than reducing error rate of a model. Still on this nonlimiting example, processor 104 may combine the metrics and generate a weighted score for the metamodel 152. The weighted score may be a weighted average where each metric is multiplied by their weight. In this example, without limitation, efficiency score 148 of metamodel 152 may be described as Efficiency score=(first process model metric*weight A)+(second process model metric*weight B). At each iteration of the comparison of first process model 124 to second process model 160 using metamodel 152, the functionality of apparatus 100 including processor 104 may be improved at least due to the improvements in the accuracy of the output generated by metamodel 152.

Still referring to FIG. 1, in an embodiment, processor 104 may be configured to receive user input. In embodiments, input data 116 may include user input. In some embodiments, user input may include at least a problem. As used herein, “a problem” is an event or situation that instills a cognitive reaction from a user. In embodiments, cognitive reaction may be related to the complexity of a problem, such as the cognitive reaction required by a user to overcome a problem. In embodiments, processor 104 may receive user input using a GUI interface. In embodiments, processor 104 may receive user input from a user device. User device may include any computing device described in this disclosure. In embodiments, user input may include textual inputs, voice inputs, audio inputs, sensor inputs, gesture recognition, file upload, selection through a GUI, and the like. In some embodiments, cognitive reaction may be related to the severity of a problem, such as the cognitive reaction related to the emotional and/or physical effects of a problem on a user. In some embodiments, user input may include problems used for deriving reaction data. In an embodiment, user input may be included in a database or any other data storage structure. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. In some embodiments, process data 112 may include user input.

Continuing to refer to FIG. 1, in some embodiments, processor 104 may be configured to generate cognitive data. As used herein, “cognitive data” is a data structure representing one or more sets of operations or activities in a human brain related to one or more events or conditions. In some embodiments, cognitive data may include data that is relevant to understanding and solving a problem. In some embodiments, processor 104 may be configured to generate cognitive data as a function of a cognition classifier. As used herein, “cognition classifier” is a machine learning model that is used to classify or categorize data based on cognitive processes or mental states. In some embodiments, cognitive classifier may include classifier described throughout this disclosure. In some embodiments, cognitive classifier may label mental states based on events or conditions. In a nonlimiting example, cognitive classifier may classify a mental state of enthusiasm to an event going on vacation. In an embodiment, cognitive data may include complexity data. As used here, “complexity data” is cognitive data related to overcoming a problem. In some embodiments, cognitive data may include severity data. As used herein, “severity data” is cognitive data related to the effects of a problem on a person's brain. In embodiments, process data 112 may include cognitive data. In some embodiments, first process model 124 may include a cognition classifier. In embodiments, second process model 160 may include cognition classifier.

Continuing to refer to FIG. 1, in some embodiments, processor 104 may be configured to determine reaction data as a function of user input and cognitive data. As used herein, “reaction data” are data structures representing correlations between cognitive responses data collected and one or more problems presented to a user for the purpose of capturing a reaction. In some embodiments, reaction data may be classified as severity reaction data and complexity reaction data. In an embodiment, severity reaction data includes reaction data related to reactions to a past event, or the ongoing effect a problem has on a user. In some embodiments, complexity reaction data may include reaction data related to the cognitive effects related to the steps required to resolve an issue or problem. In some embodiments, reaction data may include both complexity and severity labels. In a nonlimiting example, reaction data may include a data element related to a “business merger” problem, where the data element is labelled as “anticipatory stress” for complexity and “regret” for severity. In some embodiments, reaction data may be included in a database. In a nonlimiting example, a data element of reaction data may be represented as “value: ‘business merger; label1: “anticipatory stress’; label 2: ‘regret’” where lable1 corresponds to complexity and label 2 to severity. In some embodiments, process input data 116 may include user input and cognitive data. In embodiments, output data 120 may include reaction data. In some embodiments, plurality of process data 112 may include user input and cognitive data correlated to reaction data.

Still referring to FIG. 1, in some embodiments, processor 104 may be configured to determine reaction data using a reaction classifier. A “reaction classifier,” as used herein, is a machine learning algorithm used for classifying cognitive data and problems presented to reaction labels. In some embodiments, reaction classifier may be trained using training data. In some embodiments, training data may include cognitive data and user input correlated to reaction labels. Training data may include mock data generated for the purpose of training a classifier. In some embodiments, training data may include prior iterations of reaction classifier. A “reaction label,” as used herein, is a label, or category, corresponding to a reaction to a problem. In embodiments, machine learning algorithms may be used for training classifier such as decision trees, support vector machine, neural networks, and the like. In a nonlimiting example, reaction classifier may classify an input of a problem, such as a “business merger,” and cognitive data that includes severity data of the high-heart rate when presented with the “business merger” as a past event to a reaction label of “anticipatory stress” and for cognitive data that includes complexity data of high blood pressure, and other cognitive parameters related to high levels of stress, to a reaction label of “reminiscent stress.” In some embodiments, first process model 124 may include reaction classifier. In some embodiments, second process model 160 may include reaction classifier. In a nonlimiting example, processor 104 may generate process model measurement 156 for reaction classifier that includes an error metric, where generating the error metric includes comparing expected classifications included in process output data 120 to outputs of reaction classifier. In a nonlimiting example, processor 104 may utilize metamodel 152 to generate an efficiency score 148 for reaction classifier.

Still referring to FIG. 1, in some embodiments, processor 104 may be configured to generate decision training data using the reaction classifier. A “decision training data,” as used herein, are inputs and outputs pairs of a machine learning models correlated to historical data. Historical data may include past iterations of apparatus 100. In some embodiments, historical data may be inputted by users. In some embodiments, historical data may be included in a database. In some embodiments, historical data may include mock data. In embodiments, mock data may include problems presented to a user and reactions gathered from the interaction for the purpose of generating decision training data. In an embodiments, reaction classifier input/output combinations may be added to an existing decision training data. In some embodiments, existing decision training data may include a plurality of models. In some embodiments, decision training data may include previous iterations of reaction classifier. In some embodiments, processor 104 may generate decision training data using inputs/outputs of reaction classifier and add subsequent iterations of reaction classifier to decision training data. In some embodiments, decision training data may include a plurality of models with similar types of inputs/outputs correlated to historical data. In some embodiments, first process model 124 may be trained using decision training data. In some embodiments, second process model 160 may be trained using decision training data.

With continued reference to FIG. 1, in embodiments, processor 104 may be configured to generate a process metamodel as a function of decision training data. As used herein, “process metamodel,” is a model configured to improve accuracy, efficiency and interpretability of outputs of machine learning models described in this disclosure. In an embodiment, metamodel 152 may include process metamodel. In some embodiments, process metamodel may be used to identify incorrect inputs and/or outputs of machine learning models. In an embodiment, process metamodel may be used for identifying incorrect reaction labels in reaction classifier. In some embodiments, process metamodel may be used for predicting outcomes based on the multiple input and output combinations included in training data. In a nonlimiting example, process metamodel may be used to identify incorrect data generated by models by comparing inputs and output pairs of machine learning models and input/output pairs in historical data to expected outcomes. In embodiments, expected outcomes used by process metamodel may be included in historical data. In embodiments, an expected outcome may be an output included in historical data. In some embodiments, metamodel 152 may include process metamodel. In embodiments, metamodel training examples 132 may include decision training data. In some embodiments, plurality of model inputs examples 136 may include prior inputs of cognition classifier. In some embodiments, plurality of model inputs examples 136 may include prior inputs of reaction classifier. In embodiments, model output examples 140 may include prior outputs of cognition classifier. In embodiments, model output examples 140 may include prior outputs of reaction classifier. In some embodiments, exemplary model 144 may include prior iterations of cognition classifier. In some embodiments, exemplary model 144 may include prior iterations of reaction classifier. In some embodiments, metamodel 152 may receive cognition classifier and reaction classifier as inputs and output efficiency score 148 for metamodel 152. In some embodiments, metamodel 152 may generate efficiency score 148 for the cognition classifier and efficiency score 148 for reaction classifier. In a further embodiment, processor 104 may be configured to compare efficiency score 148 of reaction classifier to efficiency score 148 of cognition classifier. In a nonlimiting example, processor 104 may choose a classifier for optimization based on the efficiency score 148 comparison. It will become apparent to one of ordinary skill in the art, upon reading this disclosure, that reaction and cognition classifier are described in exemplary manner and that metamodel 152 may be used to optimize many other models not described herein.

Still referring to FIG. 1, in some embodiments, process metamodel may be used for identifying incorrect inputs and/or outputs of cognition classifier. In some embodiments, process metamodel may be used for identifying operations or activities in a human brain incorrectly correlated to events or conditions. In other embodiments, process metamodel may be used for identifying incorrect event or conditions which operations or activities in a human brain were correlated to by cognition classifier. In a nonlimiting example, cognitive classifier may classify a mental state of melancholy to an event going on vacation, where historical data includes an association of mental state of “enthusiasm” to an event going on vacation. In this nonlimiting example, process metamodel may be used to retrain cognition classifier with training data that includes the association of historical data.

Continuing to refer to FIG. 1, in an embodiment, processor 104 may be configured to identify a collateral process as a function of metamodel 152. As used herein, a “collateral process” is an alternate, or additional, output identified by metamodel 152 based on input/output combinations of models correlated to historical data. In an example, without limitations, reaction data generated by reaction classifier may include a data element related to a “business merger” problem, where the data element is labelled as “anticipatory stress” for complexity and “regret” for severity, while historical data may include other classifications related to a “business merger” problem. In this nonlimiting example, processor 104 may identify additional labels for the data element of “business merger,” such as “euphoric state” for complexity and “relief” for severity.

With continued reference to FIG. 1, in some embodiments, processor 104 may be configured to determine a reaction score. A “reaction score,” as used herein, is a quantitative measure of cognitive performance related to a condition or event. In some embodiments, input data 116 may include data related to condition or event used for determining reaction score. In embodiments, reaction score may be calculated for problems included in a user input. In some embodiments, reaction score may be calculated to problems similar to problems included in user input. In a nonlimiting example, a problem included in user input of “detrimental commute” which relates to a user's commute being so long that it affects productivity. In this nonlimiting example, processor 104 may also present user with alternatives to commute, such as “switching to a ‘work from home’ work environment,” “seeking alternate employer,” and the like. In some embodiments, reaction score may be a quantitative measure of a person's cognitive reaction to solving a problem. In embodiments, determining reaction score may include using reaction data. In some embodiments, reaction score may be a quantitative measure of the cognitive effects of a problem or event on a person. In some embodiments, processor 104 may be configured to determine reaction score as a function of cognitive data. In some embodiments, determining reaction score may include determining a complexity score. As used herein, a “complexity score” is a score related specifically to complexity data. In some embodiments, determining reaction score may include determining a severity score. A “severity score,” as used herein, is a score related specifically to severity data. In some embodiments, reaction score may be determined based on a cognitive test related to a problem. In some embodiments, reaction score may be a rating input by a user related to severity and complexity of a problem. In some embodiments, reaction score may include a complexity score and a severity score. In some embodiments, reaction score may be an average score between severity score and complexity score. In some embodiments, reaction score may be based on weighted scores for severity score and complexity score. In a nonlimiting example, score related to severity may have a weight of 40%, or 0.4, and a score related to complexity may have a score of 60%, or 0.6. In this nonlimiting example, if severity score=6 and complexity score=4, then reaction score may be represented as (0.4*6)+(0.6*4)=4.8. In some embodiments, input data 116 may include user input. In some embodiments, process output data 120 may include reaction score. In some embodiments, processor 104 may be configured to receive user input as input data 116 and generate a reaction score as a process output data 120. In a further embodiment, first process model 124 may receive user data as input data 116 and generate a reaction score as output. In further embodiments, processor 104 may generate an error metric by comparing process output data 120 that includes a reaction score to an output of first process model 124.

Still referring to FIG. 1, in some embodiments, processor 104 may be configured to identify at least a causative factor as a function of cognitive data and reaction score. A “causative factor,” as used herein, is a causal connection between cognitive data and reactions to problems or events. In some embodiments, identifying at least a causative factor may include comparing cognitive data to reaction score. In some embodiments, first process model 124 may receive cognitive data and reaction score as inputs and output a causative factor. In embodiments, processor 104 may be configured to compare cognitive data to reaction scores using a plurality of algorithms such as correlation analysis, regression analysis, cluster analysis, and the like. Processor 104 may be configured to use any algorithm described in reference to FIGS. 3-6 to identify causative factor.

Still referring to FIG. 1, in an embodiment, processor 104 may be configured to identify an input modification as a function of an alternate cognitive data and metamodel 152. An “input modification,” as used herein, is an alternate, or additional, causal connection identified by metamodel 152. In embodiments, input modification may be identified based on alternate, or additional, cognitive data identified by metamodel 152, which receives cognition classifier and historical data, included in metamodel training examples 128, as inputs. In further embodiments, processor 104 may be further configured to identify input modification by comparing alternate cognitive data to reaction scores. In a nonlimiting example, processor 104 may identify causative factor for a user, as described above, and then processor 104 may further identify alternate, or additional, root causes for issues by using process metamodel that takes cognition classifier and historical data as inputs and outputs additional, or alternate, cognitive data. Processor 104 may be configured to use any algorithm described in reference to FIGS. 3-6 to identify input modification.

Continuing to refer to FIG. 1, in some embodiments, processor 104 may be further configured to determine reaction data as a function of at least a sensor. As used herein, a “sensor” is a device configured to produce an electrical output signal for the purpose of sensing and monitoring biological events or changes in its environment. In some embodiments, sensor may include one or more processors that perform one or more processing steps as described in this disclosure. In some embodiments, at least a sensor may include, without limitations, temperature sensors, EMG sensors, ECG sensors, EEG sensors, GSR sensors, heart rate sensors, airflow sensors, pressure sensors, acoustic sensors, image sensors, magnetic field sensors, movement sensors, eye tracking sensors, and the like. In a nonlimiting example, a plurality of sensors may be placed on a user, where the sensors are configured to collect physiological responses of a user, such as brain activity and eye movements, to problems presented. In some embodiments, processor 104 may generate reaction data as a function of physiological responses collected by at least a sensor. In embodiments, processor 104 may generate process data 112 as a function of outputs form at least a sensor. In some embodiments, input data 116 may include outputs from at least a sensor. In some embodiments, generating process data 112 from sensor data may include performing sensor data preprocessing. Sensor data preprocessing may include noise reduction techniques, data normalization, signal filtering, and the like. In an embodiment, generating process data 112 from sensor output may include performing feature extraction. A “feature extraction,” as used herein, is the process of extracting relevant features or characteristics from sensor data that includes identifying patterns, thresholds and other relevant information. In some embodiments, processor 104 may use first process model 124 to perform feature extraction. In embodiments, process model measurements 152 may include extracted features. In some embodiments, processor 104 may use second process model 160 to perform feature extraction. In embodiments, generating process data 112 from sensor output may include encoding sensor data. As used herein, “encoding sensor data” is a process of transforming sensor measurements into a specific format or data structure. In embodiments, without limitation, processor 104 may encode sensor data into a plurality of formats such as JSON, XML, CSV, Binary Encoding, and the like. In some embodiments, generating process data 112 from sensor output may include performing metadata annotation. As used herein, “metadata annotation” of sensor data refers to metadata labels, or information, used for identifying the sensor data such as sensor type, calibration parameters, location of data capture, and the like. It will be apparent to one of ordinary skill in the art, upon reading this disclosure, that generating process data 112 from sensor output may include any processes or features described herein and may also include processes or features not described in this disclosure.

Still referring to FIG. 1, in some embodiments, at least a sensor may include an optical device. In some embodiments, the optical device may include at least a camera. As used in this disclosure, a “camera” is a device that is configured to sense electromagnetic radiation, such as without limitation visible light, and generate an image representing the electromagnetic radiation. In some cases, a camera may include one or more optics. Exemplary non-limiting optics include spherical lenses, aspherical lenses, reflectors, polarizers, filters, windows, aperture stops, and the like. In some cases, at least a camera may include an image sensor. Exemplary non-limiting image sensors include digital image sensors, such as without limitation charge-coupled device (CCD) sensors and complimentary metal-oxide-semiconductor (CMOS) sensors, chemical image sensors, and analog image sensors, such as without limitation film. In some cases, a camera may be sensitive within a non-visible range of electromagnetic radiation, such as without limitation infrared. As used in this disclosure, “image data” is information representing at least a physical scene, space, and/or object. In some cases, image data may be generated by a camera. “Image data” may be used interchangeably through this disclosure with “image,” where image is used as a noun. An image may be optical, such as without limitation where at least an optic is used to generate an image of an object. An image may be material, such as without limitation when film is used to capture an image. An image may be digital, such as without limitation when represented as a bitmap. Alternatively, an image may be comprised of any media capable of representing a physical scene, space, and/or object. Alternatively, where “image” is used as a verb, in this disclosure, it refers to generation and/or formation of an image.

Still referring to FIG. 1, in some embodiments, apparatus 100 may be communicatively connected to the optical device. In embodiments, the optical device may include a machine vision system that includes at least a camera. A machine vision system may use images from at least a camera, to make a determination about a scene, space, and/or object. For example, in some cases a machine vision system may be used for world modeling or registration of objects within a space. In some cases, registration may include image processing, such as without limitation object recognition, feature detection, edge/corner detection, and the like. Non-limiting examples of feature detection may include scale invariant feature transform (SIFT), Canny edge detection, Shi Tomasi corner detection, and the like. In some cases, registration may include one or more transformations to orient a camera frame (or an image or video stream) relative a three-dimensional coordinate system; exemplary transformations include without limitation homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ø may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level. In some cases, a machine vision system may use a classifier, such as any classifier described throughout this disclosure. An exemplary machine vision camera is an OpenMV Cam H7 from OpenMV, LLC of Atlanta, Georgia, U.S.A. OpenMV Cam comprises a small, low power, microcontroller which allows execution of machine vision applications. OpenMV Cam comprises an ARM Cortex M7 processor and a 640×480 image sensor operating at a frame rate up to 150 fps. OpenMV Cam may be programmed with Python using a Remote Python/Procedure Call (RPC) library. OpenMV CAM may be used to operate image classification and segmentation models, such as without limitation by way of TensorFlow Lite; detection motion, for example by way of frame differencing algorithms; marker detection, for example blob detection; object detection, for example face detection; eye tracking; person detection, for example by way of a trained machine learning model; camera motion detection, for example by way of optical flow detection; code (barcode) detection and decoding; image capture; and video recording.

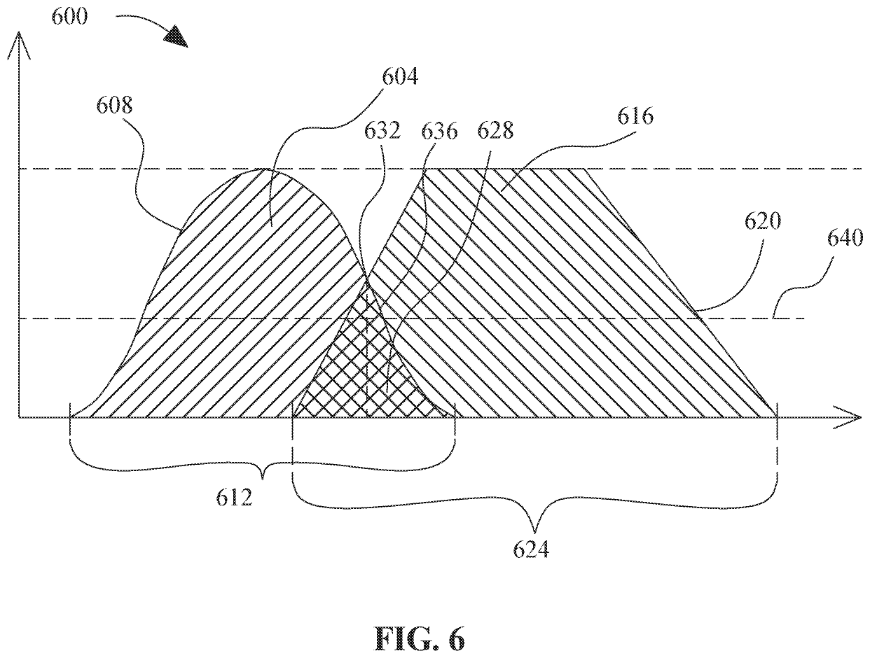

Continuing to refer to FIG. 1, in some embodiments, determining reaction score may include using a fuzzy set comparison. In an embodiment, processor 104 may define fuzzy sets for complexity of a problem and for severity of a problem. In a nonlimiting example, complexity and severity of a problem may be represented as fuzzy sets, each with a range between 0 and 1, where “0” represents low complexity or severity, and I represents high complexity or severity of a problem. In a further embodiment, processor 104 may represent reaction data as a fuzzy set with membership values representing the degree to which a user is affected by a problem. In a nonlimiting example, reaction fuzzy set may be represented by membership values ranging from 0, representing that user has not been affected by a problem, and 1, representing that user has been highly affected by a problem. In further embodiments, processor 104 may use fuzzy set comparison to determine the degree of similarity between the reaction data fuzzy set and the complexity and severity fuzzy sets, which is the degree of overlap between the fuzzy sets. In a nonlimiting example, processor may assign a weight of 30%, or 0.3, to the complexity of a problem and 70%, or 0.7, to the severity of problem. Continuing with the nonlimiting example, processor 104 may define a fuzzy set for complexity where low complexity: 0 to 0.3 membership value, medium complexity: 0.2 to 0.8 membership value and high complexity: 0.7 to 1 membership value. Continuing with the nonlimiting example, processor 104 may also define a fuzzy set for severity where low severity: 0 to 0.2 membership value, medium severity: 0.1 to 0.8 membership value and high severity: 0.7 to 1 membership value. In this nonlimiting example, processor 104 may collect reaction data, through user input or through at least a sensor 156, and represent reaction data as a fuzzy set where low reaction: 0 to 0.2 membership value, medium reaction: 0.1 to 0.8 membership value and high reaction: 0.7 to 1 membership value. Continuing on the nonlimiting example, processor 104 may then use fuzzy set comparison to calculate the degree of overlap between reaction data fuzzy set and the complexity and severity fuzzy sets. In this nonlimiting example, processor may use a “minimum” mathematical operation between two fuzzy sets. A “minimum,” as used herein, is the lowest value number within a set of numbers. Continuing with the nonlimiting example, processor 104 calculate the overlap between reaction data fuzzy and complexity fuzzy as follows, low complexity: minimum (0.2, 0.2)=0.2, medium complexity: minimum (0.1, 0.8)=0.1, and high complexity: minimum (0.7, 0.7)=0.7. Similarly, continuing with the nonlimiting example, processor 104 may calculate the overlap between reaction data fuzzy and severity fuzzy as follows, low severity: minimum (0.2, 0)=0, medium severity: (0.1, 0.8)=0.1 and high severity: minimum (0.7, 0.5)=0.5. In this nonlimiting example, processor 104 may calculate reaction sore 132 by multiplying the highest of each overlap to its weight, where fuzzy with highest weight gets selected of overlap membership is the same between complexity and severity fuzzies, and then adding all three levels, which may be represented as reaction score 132: (0.2*0.3)+ (0.1*0.7)+ (0.7*0.3)=0.34. Fuzzy set comparison and fuzzy sets are described in more detail in reference to FIG. 6.

With continued reference to FIG. 1, in an embodiment, processor 104 may be communicatively connected to a user device. In an embodiment, processor 104 may be configured to receive reaction score from user device. In some embodiments, processor 104 may be configured to receive input data 116 from user device. In some embodiments, processor 104 may be configured to transmit efficiency score 148 to a user device. A “user device,” as used herein, is a computing device capable of displaying information and receiving an input from a user. In some embodiments, user device may include at least a sensor. In some embodiments, user device may be communicatively connected to at least a sensor. In some embodiments, processor 104 may be configured to receive user input from user device. In an embodiment, processor 104 may be configured to transmit at least a causative factor to user device. In an embodiment, processor 104 may be configured to transmit collateral process to user device. In some embodiments, processor 104 may configure user device to display collateral process. In an embodiment, processor 104 may be configured to transmit input modification to user device. In some embodiments, processor 104 may configure user device to display input modification. User device may transmit a plurality of data described herein using communication protocols such as HTTP/HTTPS (HyperText Transfer Protocol), FTP (File Transfer Protocol), WebSocket, MQTT (Message querying Telemetry Transport), CoAP (Constrained Application Protocol), and the like. User device may include any computing device described throughout this disclosure. In some embodiments, processor 104 may be configured to generate a user interface. In some embodiments, the user interface may contain instructions configuring user device to display at least a causative factor. In some embodiments, the user interface may contain instructions configuring one or more computing devices to display at least a causative factor. User device may be configured to interact with a user using a chatbot. User device may include the user interface. User interface may include a graphical user interface (GUI). In some embodiments, processor 104 may generate a user interface containing instructions configuring a computing device to receive further user input as a function of at least a causative factor. In some embodiments, processor 104 may configure a computing device to receive further user input as a function of at least a causative factor using a chatbot. In a nonlimiting example, processor 104 may generate a user interface with instructions configuring a computing device, including user device, to request further information from the user after displaying at least a causative factor. In a further nonlimiting example, after identifying a root cause, causative factor, of “employment dissatisfaction” for a problem related to a work event for a user, the user interface may include instructions configuring user device to prompt user for a confirmation that user accepts the root cause identified, if the user rejects, processor 104 may configure user device, through instructions in the user interface, to receive additional user input so a new root cause can be identified. In another example, without limitations, processor 104 may configure a computing device to receive input data 116 through a chatbot, and after a root cause is identified, processor 104 may configure the computing device to request additional information using the chatbot. In some embodiments, processor 104 may generate reaction data using at least a sensor and a chatbot. In a nonlimiting example, processor 104 may prompt user, through a chatbot, to respond to a prompt by using a sensor, such as a smartphone camera, where the processor 104 may configure the smartphone camera to capture reaction of a user based on problems displayed to the user on a screen. In some embodiments, processors 104 may capture reaction data through a user interface and at least a sensor. In a nonlimiting example, processor 104 may generate a user interface, where the user interface includes instructions configuring a computing device, such as a smartphone, to display problems to a user and further configuring a sensor included in computing device, such as a smartphone camera, to capture reactions of the user to the problems displayed. In an embodiment, processor 104 may be configured to receive metamodel training examples 128 from user device. In some embodiments, processor 104 may be configured to receive plurality of process input data 116 from user device.

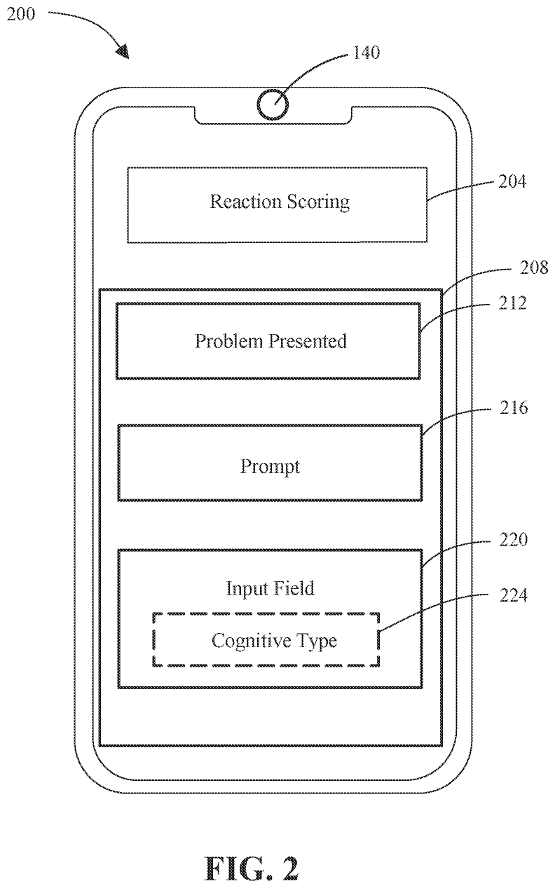

Now referring to FIG. 2, an exemplary embodiment of a user reaction interface 200 is presented. In embodiments, user reaction interface 200 may include instructions configuring a computing device to display and receive data. In embodiments, computing device may include any device capable of displaying a Graphical User Interface (GUI). Computing device may be communicatively connected to processor 104. Computing device may include a user device. In an embodiment, user reaction interface 200 may be a Graphical User Interface (GUI). In embodiments, computing device may be configured to display any data structure described throughout this disclosure. In nonlimiting examples, computing device may be a smartphone, smartwatch, laptop, desktop computer, virtual reality headset and the like. In embodiments, computing device may include a display. Display may include any display capable of presenting information in graphical form. In embodiments, Display may be configured to display a GUI. In embodiments, computing device may include a plurality of displays. In embodiments, display is configured to display a window. A “window,” as used herein, is a graphical representation that occupies at least a position of the display. In embodiments, window may occupy the entire display. In some embodiments, display may be communicatively connected to computing device. In some embodiments, a window may include one or more tabs. A “tab,” as used herein, is a subsection of a window. In a nonlimiting example, computing device may transmit information to display. In an example, without limitations, a user may navigate through a first second, third and fourth window (and so on) by interacting with display. In another nonlimiting example, a user may navigate through a first second, third and fourth window (and so on) by interacting directly with computing device, and indirectly with display, such as when information is being transmitted to a remote display. may further contain event handlers, wherein the placement of text within a textbox may signify to computing device, or display, to display another window. An “event handler” as used in this disclosure is a callback routine that operates asynchronously once an event takes place. Event handlers may include, without limitation, one or more programs to perform one or more actions based on user input, such as generating pop-up windows, submitting forms, requesting more information, and the like. For example, an event handler may be programmed to request more information or may be programmed to generate messages following a user input. User input may include clicking buttons, mouse clicks, hovering of a mouse, input using a touchscreen, keyboard clicks, an entry of characters, entry of symbols, an upload of an image, an upload of a computer file, manipulation of computer icons, and the like. For example, an event handler may be programmed to generate a notification screen following a user input wherein the notification screen notifies a user that the data was properly received. In some embodiments, an event handler may be programmed to request additional information after a first user input is received. In some embodiments, an event handler may be programmed to generate a pop-up notification when a user input is left blank. In some embodiments, an event handler may be programmed to generate requests based on the user input. In some embodiments, an event handler may be configured to capture data generated by at least a sensor 156. In a nonlimiting example, event handler could be triggered when user interfaces with an input function of user reaction interface 200, where user reaction interface 200 may contain instructions configuring a camera communicatively connected to computing device to capture a user's facial expressions while interacting with user reaction interface 200. In an embodiment, user reaction interface 200 may contain instructions configuring a computing device to display a section heading 204. A “section heading,” as used herein, is portion of user reaction interface 200 that conveys to a user what section of an application the user is interacting with. In a nonlimiting example, section heading 204 may include a textual field that describes the sections as “Reaction Scoring.” In some embodiments, user reaction interface 200 may contain instructions configuring a computing device to display a user interaction section 208. A “user interaction section,” as used herein, is a section displayed by computing device that is also configured to receive user input. In some embodiments, user interaction section 208 may include a topic sub-heading 212. In a nonlimiting example, topic sub-heading 212 may include a text-field with a problem to which a reaction is by the user is sought. In some embodiments, user interaction section 208 may include a reaction prompt sub-heading 216. In embodiments, reaction prompt sub-heading 216 may include details on how to input a reaction. In embodiments, user interaction section 208 may include an input field 220. In a nonlimiting example, input filed 220 may include a text filed where the user may input text. In other examples, input field 220 may further include a cognitive type option 224. In some embodiments, cognitive type option 224 may include option selections, such as through radio buttons or checkmark boxes, for a user to select to which cognitive type a reaction applies to. In a nonlimiting example, cognitive type option may include buttons to select between complexity type and severity type. In further embodiments, an input into user interaction section 208 may trigger capture of data from at least a sensor. In a nonlimiting example, user reaction interface 200 may configure a computing device to prompt a user for a reaction to a problem by presenting a set of options for reaction. In a further nonlimiting example, user reaction interface 200 may configure computing device to capture data from the user through at least a sensor, such as capturing video data, while user is interfacing with user interaction section. In this nonlimiting example, processor 104 may collect reaction data from the user though sensors while user is making a selection related to a problem. In some embodiments, processor 104 may be configured to use both the sensor data and the input data as reaction data. In some embodiments, processor 104 may only use sensor data as reaction data, where the prompt for user input is only used to capture a user reaction to a problem presented. In some embodiments, data from sensor and user input may be used as training data for reaction classifier. In some embodiments, processor 104 may configure user reaction interface 200 to display collateral process to user. In some embodiments, processor 104 may configure user reaction interface 200 to display input modification. In a nonlimiting example, user reaction interface 200 may be configured to display a suggested input for the user based on prior interaction with apparatus 100, such as displaying a suggested input inside the reaction prompt sub-heading 216.

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example, need for transportation may be an input and a closely associated goal, such as financing a vehicle, may be an output.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described in this disclosure as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

With continued reference to FIG. 3, machine-learning algorithms may include, without limitations, a metamodel. A metamodel, as used herein, is a set of concepts that describe elements, relationships and rules governing a specific machine learning model. In an embodiment, a metamodel may be a machine-learning model of another machine-learning model. Metamodels may include any machine-learning model algorithm described in this disclosure. Examples of machine-learning algorithms may include decision trees, neural networks, Bayesian networks, genetic algorithms, support vector machines (SVM), reinforcement learning, and the like.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

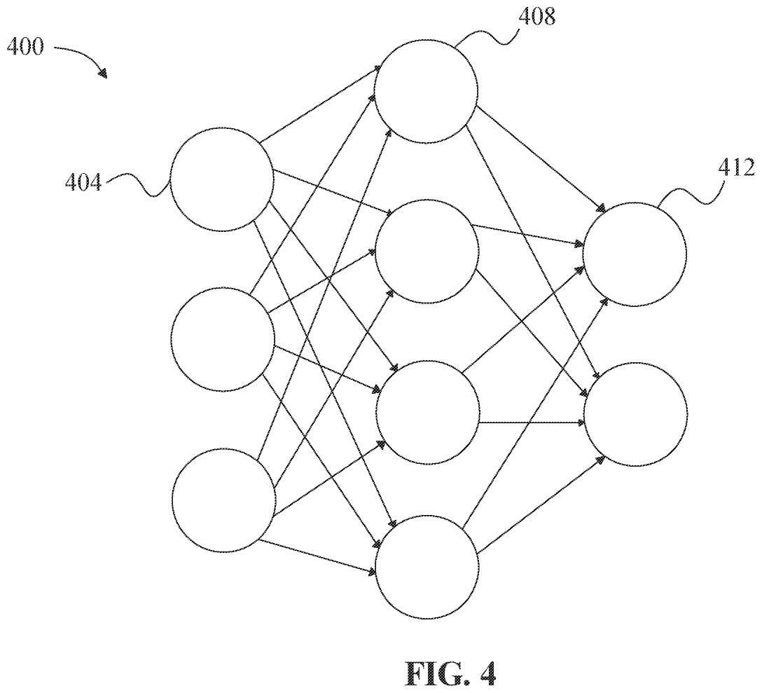

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

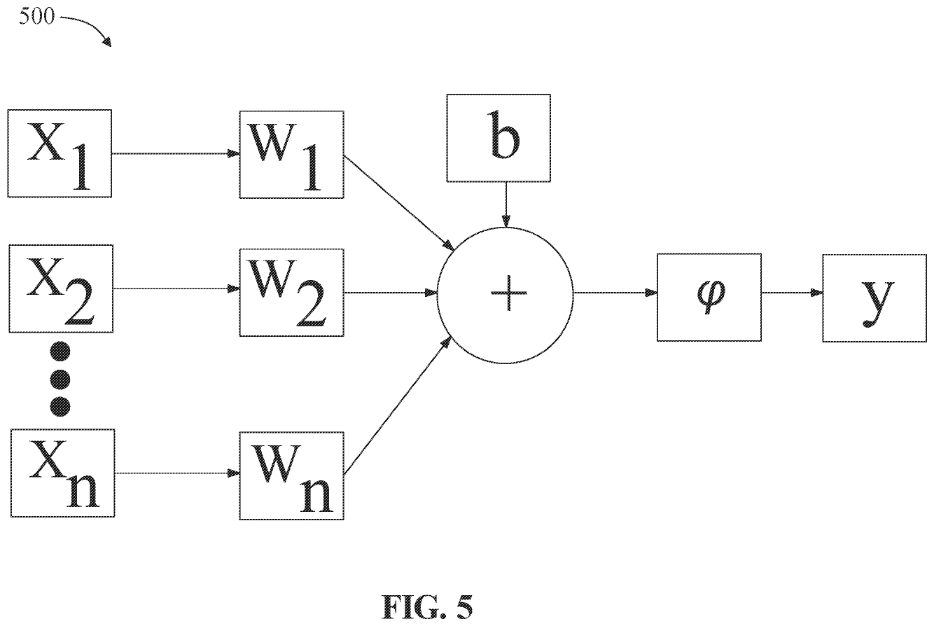

Referring now to FIG. 5, an exemplary embodiment of a node 500 of a neural network is illustrated. A node may include, without limitation, a plurality of inputs xi that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xi that may be used as activation functions. As a non-limiting and illustrative example, node may perform a weighted sum of inputs using weights w; that are multiplied by respective inputs xi. Additionally or alternatively, a bias b may be added to the weighted sum of the inputs such that an offset is added to each unit in the neural network layer that is independent of the input to the layer. The weighted sum may then be input into a function o, which may generate one or more outputs y. Weight wᵢ applied to an input xi may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Referring to FIG. 6, an exemplary embodiment of fuzzy set comparison 600 is illustrated. A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 6, first fuzzy set 604 may represent any value or combination of values as described above, including output from one or more machine-learning models. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 628 that overlaps, first membership function 608 and second membership function 620 may intersect at a point 632 representing a probability, as defined on probability interval, of a match between first fuzzy set 604 and second fuzzy set 616. Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, threshold may indicate a sufficient degree of overlap between an output from one or more machine-learning models. Alternatively or additionally, each threshold may be tuned by a machine-learning and/or statistical process, for instance and without limitation as described in further detail below.

Further referring to FIG. 6, in an embodiment, a degree of match between fuzzy sets may be used to classify a reaction. For instance, if a reaction has a fuzzy set matching a reaction associated with a cognitive fuzzy set by having a degree of overlap exceeding a threshold, processor 104 may classify the reaction as belonging to a cognitive type. Such as complexity type or severity type. Where multiple fuzzy matches are performed, degrees of match for each respective fuzzy set may be computed and aggregated through, for instance, addition, averaging, or the like, to determine an overall degree of match.

Still referring to FIG. 6, in an embodiment, a reaction may be compared to multiple cognitive fuzzy sets. For instance, reaction may be represented by a fuzzy set that is compared to each of the multiple cognitive fuzzy sets; and a degree of overlap exceeding a threshold between the reaction fuzzy set and any of the multiple cognitive fuzzy sets may cause apparatus 100 to classify the reaction as belonging to a cognitive type. For instance, in one embodiment there may be two cognitive fuzzy sets, representing respectively a complexity type and a severity type. Complexity type may have a complexity fuzzy set; severity type may have a severity fuzzy set; and reaction may have a reaction fuzzy set. Processor 104, for example, may compare a reaction fuzzy set with each of complexity fuzzy set and severity fuzzy set, as described above, and classify a condition to either, both, or neither of complexity nor severity. Machine-learning methods as described throughout may, in a non-limiting example, generate coefficients used in fuzzy set equations as described above, such as without limitation x, c, and o of a Gaussian set as described above, as outputs of machine-learning methods. Likewise, reaction may be used indirectly to determine a fuzzy set, as reaction fuzzy set may be derived from outputs of one or more machine-learning models that take the reactions directly or indirectly as inputs.

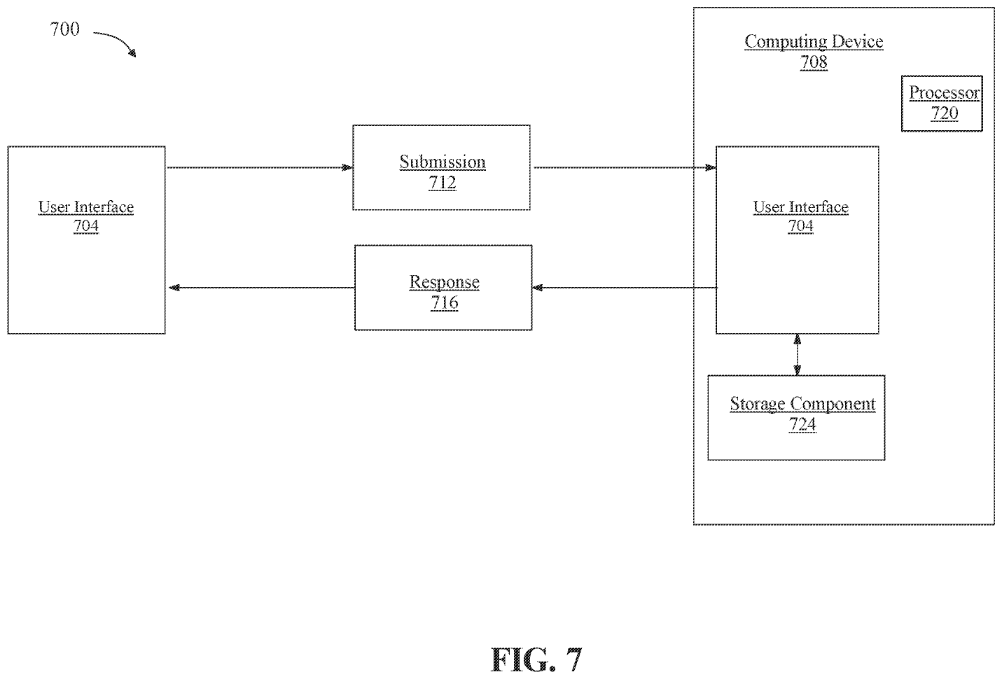

Now referring to FIG. 7, an illustrative embodiment of a chatbot system 700 is presented. According to some embodiments, a user interface 704 may be communicative with a computing device 708 that is configured to operate a chatbot. In some cases, user interface 704 may be local to computing device 708. Alternatively or additionally, in some cases, user interface 704 may remote to computing device 708 and communicative with the computing device 708, by way of one or more networks, such as without limitation the internet. In some embodiments, computing device 708 may include user device 160. In embodiments, computing device 708 may be communicatively connected to processor 104. Alternatively or additionally, user interface 704 may communicate with computing device 708 using telephonic devices and networks, such as without limitation fax machines, short message service (SMS), or multimedia message service (MMS). Commonly, user interface 704 communicates with computing device 708 using text-based communication, for example without limitation using a character encoding protocol, such as American Standard for Information Interchange (ASCII). Typically, a user interface 704 conversationally interfaces a chatbot, by way of at least a submission 712, from the user interface 704 to the chatbot, and a response 716, from the chatbot to the user interface 704. In many cases, one or both of submission 712 and response 716 are text-based communication. Alternatively or additionally, in some cases, one or both of submission 712 and response 716 are audio-based communication.

Continuing in reference to FIG. 7, a submission 712 once received by computing device 708 operating a chatbot, may be processed by a processor 720. In some embodiments, processor 720 processes a submission 712 using one or more of keyword recognition, pattern matching, and natural language processing. In some embodiments, processor employs real-time learning with evolutionary algorithms. In some cases, processor 720 may retrieve a pre-prepared response from at least a storage component 724, based upon submission 712. Alternatively or additionally, in some embodiments, processor 720 communicates a response 716 without first receiving a submission 712, thereby initiating conversation. In some cases, processor 720 communicates an inquiry to user interface 704; and the processor is configured to process an answer to the inquiry in a following submission 712 from the user interface 704. In a nonlimiting example, an answer to an inquiry presented within a submission 712 from user device 160 may be used by processor 104 as an input to another function, for example without limitation a follow-up question.

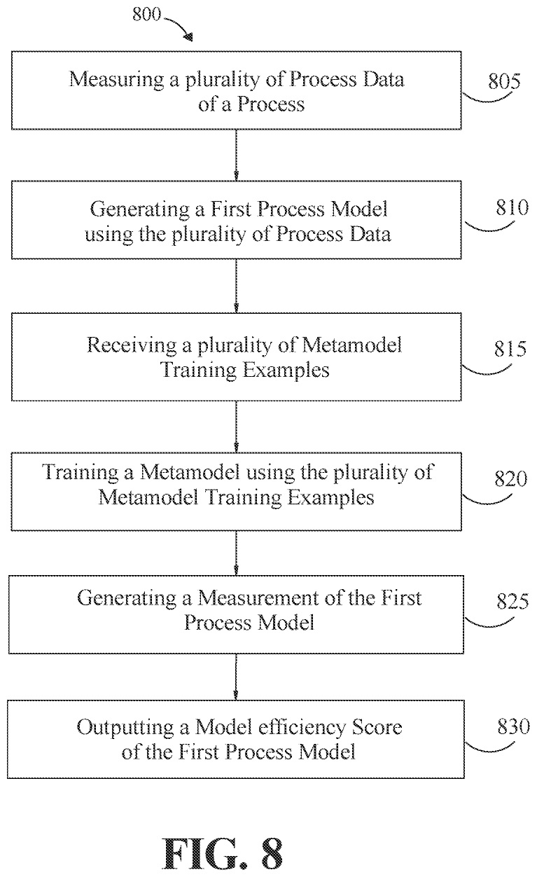

Now referring to FIG. 8, an exemplary embodiment of a method 800 for the identifying collateral processes is presented. In an embodiment, method 800, at step 805, includes measuring plurality of process data 112, wherein plurality of process data 112 includes plurality of process input data 116 and plurality of correlated process output data 120. In some embodiments, method 800 may further include receiving a user input. In some embodiments, method 800 may include receiving user input using a chatbot. In some embodiments, method 800 may include receiving user input from user device 160. Method 800 may be consistent with any disclosure in reference to FIGS. 1-7.

Continuing to refer to FIG. 8, method 800, at step 810, in an embodiment, includes generating first process model 124 using plurality of process data 112, wherein generating first process model 124 further includes training first process model 124 using plurality of process input data 116 and plurality of correlated process output data 120. In some embodiments, method 800 may include generating cognitive data 116. In embodiments, method 800 may include generating cognitive data 116 as a function of a cognition classifier 120. In embodiments, cognition classifier may label an element of cognitive data 116 as a severity type. In other embodiments, cognition classifier 120 may classify an element of cognitive data 116 as “other type.” A complexity type is an element of cognitive data 116 that is labelled as being complexity data 124. A severity type is an element of cognitive data 116 that is labelled as being severity data 128. An other type is an element of cognitive data 116 that does not belong to either complexity data 124 or severity data 128. In a nonlimiting example, an input received from a user interaction with user reaction interface may be generated as an element of cognitive data 116 but may be labeled as “other type” because the emotional reaction does not apply to the problem presented to the user. In another nonlimiting example, an input received from a user interaction with a user reaction interface may be generated as an element of cognitive data 116 and labelled as belonging to complexity data 214 if the user reaction is related to a reaction to steps for resolving the problem presented. An exemplary embodiment of user reaction interface is described in more detail in reference to FIG. 2. Method 800 may be consistent with any disclosure in reference to FIGS. 1-7.

With continued reference to FIG. 8, in some embodiments, at step 815, method 800 includes receiving plurality of metamodel training examples 128, wherein each metamodel training example 132 includes plurality of model input examples 136 and plurality of model output examples 140 corresponding to an exemplary model 144. In some embodiments, method 800 may include determining reaction data 124 using reaction classifier 128. In some embodiments, method 800 may include determining reaction score 144. In some embodiments, method 800 may include determining reaction score 144 as a function of a reaction score machine learning model. In an embodiment, method 800 may include determining reaction score 144 comprises using a fuzzy set comparison. In some embodiments, method 800 may include determining reaction score 144 using at least a sensor 156. In some embodiments, at least a sensor 156 may include a wearable device. In some embodiments, at least a sensor 156 may include an eye tracking sensor. In some embodiments, method 800 may include determining reaction score 144 as a function of tracking eye movements of a user. In some embodiments, method 800 may further include generating reaction data as a function of user input. In some embodiments, method 800 may include determining reaction score 144 using reaction data. In some embodiments, method 800 may include generating reaction data as a function of at least a sensor 156. Method 800 may include any embodiments described in in reference to FIGS. 1-7.

With continued reference to FIG. 8, in some embodiments, at step 820, method 800 includes training metamodel 152 using plurality of metamodel training examples 128, wherein metamodel is configured to receive process model measurements 156 as input and output model efficiency scores 148. In an embodiment, method 800 may include generating decision training data 132 using reaction classifier 128. Method 800 may include any embodiments described in in reference to FIGS. 1-7.

With continued reference to FIG. 8, at step 825, in an embodiment, method 800 includes generating process metamodel 136 as a function of decision training data 132. In embodiments, method 800 may include identifying at least a causative factor 148 as a function of cognitive data 116 and reaction score 144. In an embodiment, identifying at least a causative factor 148 may include comparing cognitive data 116 to reaction score 144. In some embodiments, identifying at least a causative factor 148 may include using a machine learning model. Method 800 may be consistent with any disclosure in reference to FIGS. 1-7.

Still referring to FIG. 8. In embodiments, method 800, at step 830, includes outputting model efficiency score 148 of first process model 124 using metamodel 152 and measurement 156 of first process model 124. In embodiments, method 800 may include identifying at least a collateral process 140 as a function of process metamodel 136. In embodiments, method 800 may further include identifying input modification 152. In embodiments, identifying input modification 152 may include generating alternate cognitive data 116 as a function of cognitive classifier 120 and process metamodel 136. In further embodiments, identifying input modification 152 may further include comparing the alternate cognitive data 116 to reaction score 144.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

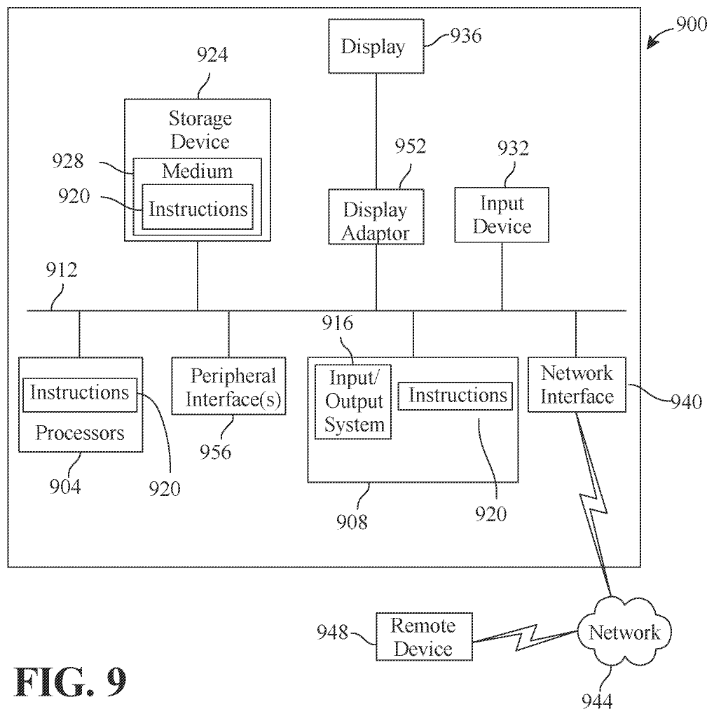

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for identifying collateral processes comprising:
a plurality of sensors, wherein the plurality of sensors are placed on a user;
at least a processor; and
a memory communicatively connected to the at least the processor, the memory containing instructions configuring the processor to:
collect, using the plurality of sensors, physiological responses of the user;
generate reaction data as a function of the collected physiological responses of the user;
generate a plurality of process data of a process as a function of outputs from the plurality of sensors, wherein the plurality of process data further comprises a plurality of process input data and a plurality of correlated process output data, wherein the plurality of process input data comprises cognitive data correlated to reaction data, wherein the cognitive data comprises complexity data, wherein the complexity data comprises cognitive data related to overcoming a problem, wherein generating the plurality of process data further comprises:
performing feature extraction on the physiological responses of the user collected using the plurality of sensors;
generate the cognitive data as a function of a cognition classifier; and
determine the reaction data as a function of a user input and the cognitive data;
generate a first process model using the plurality of process data, wherein generating the first process model further comprises training the process model using the plurality of process input data and the plurality of correlated process output data;
receive a plurality of metamodel training examples, wherein each metamodel training example of the plurality of metamodel training examples includes a plurality of model input examples and model output examples corresponding to an exemplary model, and wherein each model training example within the plurality of metamodel training examples further includes at least an efficiency score per the exemplary model relating to the plurality of model input examples and model output examples;
train, using the plurality of metamodel training examples, a metamodel, wherein the metamodel is configured to receive process model measurements and output model efficiency scores;
generate a measurement of the first process model;
output a model efficiency score of the first process model using the metamodel and the measurement of the first process model;
identify a collateral process as a function of the metamodel wherein the collateral process indicates an additional output identified by the metamodel as a function of the plurality of metamodel training examples correlated to historical data; and
transmit the collateral process to a user device.

2. The apparatus of claim 1, wherein the processor is further configured to generate a second process model.

3. The apparatus of claim 2, wherein the processor is further configured to compare the second process model to the first process model using the metamodel.

4. The apparatus of claim 1, wherein the processor is further configured to:
determine a reaction score as a function of the cognitive data and the user input; and
identify at least a causative factor as a function of the cognitive data and the reaction score, wherein identifying the at least a causative factor further comprises comparing the cognitive data to the reaction score.

5. The apparatus of claim 4, wherein the processor is further configured to generate an input modification, wherein generating the input modification further comprises:
generating alternate cognitive data as a function of a cognitive classifier and the metamodel; and
comparing the alternate cognitive data to the reaction score.

6. (canceled)

7. The apparatus of claim 1, wherein the plurality of the sensors comprises a wearable device.

8. The apparatus of claim 1, wherein the plurality of sensors comprises an eye tracking sensor.

9. The apparatus of claim 8, wherein the processor is further configured to generate the reaction data as a function of tracking eye movements of a user.

10. The apparatus of claim 1, wherein the processor is further configured to receive the user input using a chatbot.

11. A method for identifying collateral processes, the method comprising:
collecting, using a plurality of sensors, physiological responses of a user;
generating, by at least a processor, a plurality of process data of a process, wherein the plurality of process data further comprises a plurality of process input data and a plurality of correlated process output data, wherein the plurality of process input data comprises cognitive data correlated to reaction data, wherein the cognitive data comprises complexity data, wherein the complexity data comprises cognitive data related to overcoming a problem, wherein generating the plurality of process data further comprises:
performing feature extraction on the physiological responses of the user collected using the plurality of sensors;
generating, by the at least the processor, the cognitive data as a function of a cognition classifier;
determining, by the at least the processor, the reaction data as a function of a user input and the cognitive data;
generating, by the at least the processor, a first process model using the plurality of process data, wherein generating the first process model further comprises training the process model using the plurality of process input data and the plurality of correlated process output data;
receiving, by the at least the processor, a plurality of metamodel training examples, wherein each metamodel training example of the plurality of metamodel training examples includes a plurality of model input examples and model output examples corresponding to an exemplary model, and wherein each metamodel training example within the plurality of metamodel training examples further includes at least an efficiency score per the exemplary model relating to the plurality of model input examples and model output examples;
training, by the at least the processor, a metamodel using the plurality of metamodel training examples, wherein the metamodel is configured to receive process model measurements and output model efficiency scores;
generating, by the at least the processor, a measurement of the first process model;
outputting, by the at least the processor, a model efficiency score of the first process model using the metamodel and the measurement of the first process model;
identifying, by the at least the processor, a collateral process as a function of the metamodel wherein the collateral process indicates an additional output identified by the metamodel as a function of the plurality of metamodel training examples correlated to historical data; and
transmitting, by the at least the processor, the collateral process to a user device.

12. The method of claim 11, wherein the method further comprises generating a second process model.

13. The method of claim 12, wherein the method further comprises comparing the second process model to the first process model using the metamodel.

14. The method of claim 11, wherein the method further comprises:
determining, by the at least the processor, a reaction score as a function of the cognitive data and the user input; and
identifying, by the at least the processor, at least a causative factor as a function of the cognitive data and the reaction score, wherein identifying the at least a causative factor further comprises comparing the cognitive data to the reaction score.

15. The method of claim 14, wherein the method further comprises generating an input modification, wherein generating the input modification further comprises:
generating alternate cognitive data as a function of a cognitive classifier and the metamodel; and
comparing the alternate cognitive data to the reaction score.

16. (canceled)

17. The method of claim 11, wherein the plurality of sensors comprises a wearable device.

18. The method of claim 11, wherein the plurality of sensors comprises an eye tracking sensor.

19. The method of claim 18, wherein the method further comprises generating the reaction data as a function of tracking eye movements of a user.

20. The method of claim 11, wherein the method further comprises receiving the user input using a chatbot.
